(19) United States
(12) Patent Application Publication
(10) Pub. No.: US 2019/0284630 A1
(43) Pub. Date: Sep. 19, 2019

(54) RETINAL GANGLION CELL SUBTYPE DIFFERENTIATION FROM HUMAN PLURIPOTENT STEM CELLS

(71) Applicant: Indiana University Research and Technology Corporation, Indianapolis, IN (US)

(72) Inventors: Jason S. Meyer, Indianapolis, IN (US); Kirstin Brooke VanderWall, Indianapolis, IN (US)

(21) Appl. No.: 16/354,888

(22) Filed: Mar. 15, 2019

Related U.S. Application Data
(60) Provisional application No. 62/644,010, filed on Mar. 16, 2018.

(51) Int. Cl. C12Q 1/6883; C12N 5/0793

(52) U.S. Cl. CPC C12Q 1/6883; C12N 2506/03; C12Q 2600/158; C12N 5/0619

(57) ABSTRACT

In certain aspects, the present disclosure provides methods and materials for detection and characterization of retinal ganglion cells in a sample. In accordance with certain embodiments, the present disclosure provides systems and methods for detection of disease or diseased states related to retinal ganglion cells. In some forms the disclosure provides for a method for diagnosing a disease or diseased state related to retinal ganglion cells in a patient, the method comprising the step of detecting in a body fluid of the patient one or more markers associated with the disease or diseased state.

### CROSS-REFERENCE TO RELATED APPLICATION

This application claims the benefit of U.S. Provisional Application No. 62/644,010 filed Mar. 16, 2018, which is hereby incorporated by reference.

### STATEMENT OF GOVERNMENTAL RIGHTS

This invention was made with government support under EY024984 awarded by National Institutes of Health. The government has certain rights in the invention.

### BACKGROUND

Retinal ganglion cells (RGCs) are the projection neurons of the visual system responsible for transmission of signals from the retina to the brain along their axons within the optic nerve. While long distance connectivity with post-synaptic targets is common to all RGCs, these cells may differ in their physiological roles exhibiting varied responses to visual stimuli. Traditionally, these functional differences among RGCs have been characterized by physiological parameters as well as variation in their dendritic arborization within the inner plexiform layer. More recently, a number of molecular markers have been described to further classify different subtypes of RGCs, with more than 30 different subtypes identified to date. Previous efforts have identified numerous RGC subtypes in animal models, but less attention has been paid to human RGCs.

Many previous studies focused upon RGC subtypes have relied upon the use of animal models, leading to the ability to identify these cells and study their functional characteristics. However, the study of RGC subtypes in the human system has been lacking, due to the limited availability of adult tissue and inaccessibility of the human retina at early developmental stages. Human pluripotent stem cells (hPSCs) provide a powerful tool for studies of cell type diversity, as they have the ability to self-renew and give rise to all cell types of the body. Many previous efforts have examined the ability of hPSCs to give rise to RGCs. However, this differentiation has focused upon the generation of retinal ganglion cells as a whole, without a focus on the numerous subtypes of RGCs that are known to exist. A need therefore exists for the study of the cellular mosaicism that exists among RGCs of the human retina, with important implications for studies of how these cells differ in their function as well as how they may be affected in disease states.

### SUMMARY

In certain aspects, the present disclosure provides methods and materials for detection and characterization of retinal ganglion cells in a sample. In accordance with certain embodiments, the present disclosure provides systems and methods for detection of disease or diseased states related to retinal ganglion cells. In some forms the disclosure provides for a method for diagnosing a disease or diseased state related to retinal ganglion cells in a patient, the method comprising the step of detecting in a body fluid of the patient one or more markers associated with the disease or diseased state. In some forms the patient is a mammal, for example a human. In certain embodiments the marker indicates ON-OFF direction selective retinal ganglion cells. In certain embodiments marker comprises BRN3 co-expressed with at least one of CART or CDH6. In certain embodiments the marker indicates ON direction selective retinal ganglion cells. In certain embodiments the marker comprises FSTL4 co-localized with BRN3. In certain embodiments the marker indicates alpha retinal ganglion cells. In certain embodiments the marker comprises BRN3 co-localized with at least one of SPP1 or CB2. In certain embodiments the marker indicates intrinsically photosensitive retinal ganglion cells. In certain embodiments the marker comprises Melanopsin. In certain embodiments the marker comprises DCX. In certain embodiments the marker comprises DCX co-localized with FSTL4.

In accordance with some forms the present disclosure provides a method for detection of retinal ganglion cells in a cell culture, the method comprising the steps of: preparing a cell culture of human pluripotent stem cells; and detecting one or more markers for retinal ganglion cells in the cell culture. In certain embodiments the marker comprises BRN3 co-expressed with at least one of CART or CDH6. In certain embodiments the marker comprises FSTL4 co-localized with BRN3. In certain embodiments the marker comprises BRN3 co-localized with at least one of SPP1 or CB2. In certain embodiments the marker comprises Melanopsin. In certain embodiments the marker comprises DCX. In certain embodiments the marker comprises DCX co-localized with FSTL4.

Additional embodiments, as well as features and advantages of embodiments of the invention, will be apparent from the description herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIG. 1: hPSC-derived RGCs display elaborate morphologies and diversity of gene expression. (A) DIC imaging demonstrated morphological characteristics of hPSC-derived RGCs with large three-dimensional cell bodies, projecting numerous lengthy neurites. (B-D) immunocytochemistry confirmed the RGC identity of these cells through the expression of specific markers such as BRN3, ISL1, and SNCG, as well as the extension of long processes indicated by cytoskeletal markers. (E-1) Additionally, analysis of RGC markers revealed varying degrees of co-expression within the RGC population, (J) Quantification of immunocytochemistry results verified variation in RGC-associated gene expression among hPSC-derived RGCs. Scale bars equal 50 μm in A-D, 25 μm in E-1, scale bar in B applies to B-D, scale bar in E applies to E-1. Error bars represent standard error of the mean (n=27 technical replicates from 3 biological replicates for each bar using miPS2, H9, and H7 cell lines).

FIG. 2: hPSC derived RGCs demonstrate unique transcriptional profiles. (A-C) Spearman's Rank Correlation Coefficient Analysis (SRCCA) was performed on single cell RNAseq data using RGC-specific target genes. Cells were ranked from highest to lowest expression and transformed into z-scores. Results were constructed into a heat map, with the top 20 correlating genes shown for each RGC marker, (Corresponding color key histograms for A-C are displayed in a-c). (D) The combination of SRCCA from four RGC target genes for the top 200 correlating genes revealed differential gene expression as well as a core set of 11 genes highly expressed within RGCs. n=3 biological replicates using the H9 cell line.

FIG. 3: hPSCs give rise to multiple DS-RGC subtypes. (A-B)ON-OFF DS-RGCs were identified by the co-expression of the RGC-marker BRN3 with either CART or CDH6. (C) ON DS-RGCs were identified by the expression of FSTL4 co-localized with BRN3. (D) Quantification of immunocytochemistry results identified the expression of CART, CDH6, and FSTL4 within the BRN3-RGC population at 25.95%±0.46%, 17.16%±0.51%, and 30.49%±0.58%, respectively. (E) Single cell qRT-PCR analysis demonstrated the combinatorial expression of DS-RGC markers, in conjunction with RGC-associated markers. Error bars represent the standard error of the mean (n=36 technical replicates from 3 biological replicates for each bar using miPS2, H9, and H7 cell lines). Scale bars equal 20 μm.

FIG. 4: Identification of alpha RGCs in hPSC-derived RGCs (A-B) Alpha-RGCs were identified by the co-localization of BRN3 with either SPP1 or CB2. (C) Quantification of immunocytochemistry results indicated SPP1 and CB2 were co expressed with BRN3 in 21.10%±0.42% and 15.72%±0.45% of all cells, respectively. (D) Single cell qRT-PCR analyses demonstrated the combinatorial expression of alpha RGC markers, along with the expression of pan-RGC markers. Error bars represent the standard error of the mean (n=36 technical replicates from 3 biological replicates for each bar using miPS2, H9, and H7 cell lines). Scale bars equal 30 μm.

FIG. 5: Characterization of intrinsically photosensitive-RGCs derived from hPSCs (A-B) A subset of hPSC-derived cells exhibited the expression of Melanopsin co-expressing either with or without BRN3. (C) Quantification of immunocytochemistry results was performed as a percentage of the total DAP1-positive population and Melanopsin-positive/BRN3-negative cells comprised 2.75%±0.13% of all cells, while Melanopsin-positive/BRN3-positive comprised 1.17%±0.06% of all cells. (D) Single cell qRT-PCR revealed the combinatorial expression of Melanopsin with a variety of other RGC related markers, Error bars represent the standard error of the mean (n=36 chemical replicates from 3 biological replicates for each bar using miPS2, H9, and H7 cell lines). Scale bar equals 50 μm.

FIG. 6: Identification of novel DS-associated markers using single cell RNAseq analysis. (A) SRCCA from FSTL4, BRN3B, and SNCG were combined for the top 1000 correlating genes and 148 genes were found to be commonly expressed between the three populations. (B-D) Additionally, SRCCA for FSTL4 was combined with retinal progenitor genes, RPE genes, and photoreceptor genes and demonstrated minimal overlapping expression. n=3 biological replicates using the H9 cell line.

FIG. 7: Identification and confirmation of DCX as a novel DS-RGC marker. (A-C) DCX was highly co-localized with FSTL4, while its co-expression with pan-RGC markers BRN3 and SNCG demonstrated less correlation. (D) Quantification of immunocytochemistry results indicated that DCX expression correlated with 82.48%±1.66% of FSTL4-positive RGCs, while it was identified in subsets of BRN3- and SNCG-positive RGCs at 42.61%±1.88% and 53.57%±1.88%, respectively. (E) Single cell RNAseq values demonstrate expression of DCX correlated with other DS-RGC markers, but was found exclusive of markers of other RGC subtypes and retinal cells. Scale bars equal 50 μm. Error bars represent standard error of the mean (n=30 technical replicates from 3 biological replicates for each bar using miPS2, H9, and H7 cell lines).

FIG. 8: Single cell qRT-PCR reveals expression of markers for other RGC subtypes. A variety of other subtypes were characterized using single cell qRT-PCR. (A) PV-, W3B-, J-RGCs were identified through the expression of PVALB, SDK2, and JAMB respectively, in addition to their combinatorial expression with a variety of other RGC-associated markers.

FIG. 9: Single cell RNA-seq analyses indicate correlative genes for major RGC subtype classes. Single cell RNAseq analyses of numerous subtypes reveals closely correlated genes. (A-D) SRCCA for BRN3B using the top 1000 correlating genes was combined with SRCCA for subtype specific markers and revealed correlated genes. For ip-. Alpha-, W3B-, and On OFF DS-RGCs 77, 23, 9, and 6 genes were found closely correlated within the top 1000 genes.

FIG. 10 is a table indicating the major classes of RGC subtypes and molecular markers for each.

FIG. 11 is a table representing information for primary antibodies used throughout the study described herein.

### DESCRIPTION

Where the definition of a term departs from the commonly used meaning of the term, applicant intends to utilize the definitions provided below, unless specifically indicated otherwise.

It is to be understood that the foregoing general description and the following detailed description are exemplary and explanatory only and are not restrictive of any patient matter claimed. In this application, the use of the singular includes the plural unless specifically stated otherwise. It must be noted that, as used in the specification and the appended claims, the singular forms “a”, “an”, and “the” include plural referents unless the context clearly dictates otherwise.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “comprising”, the term “having”, the term “including,” and variations of these words are intended to be open-ended and mean that there may be additional elements other than the listed elements.

The term “patient” or “patient” as used herein, refers to an animal which is the object of treatment, observation or experiment. By way of example only, a patient may be, but is not limited to, a mammal including, but not limited to, a human.

The present disclosure relates to the identification and molecular characterization of multiple RGC subtypes derived from hPSCs. As used herein the term human Pluripotent Stem Cells (HPSCs) encompasses human induced Pluripotent Stem Cells. Numerous RGC subtypes were identified within cultures of hPSC-derived RGCs based upon their expression of characteristic genes, including the combinatorial expression of molecular markers allowing for their classification into a proper subtype class. The results of this study are the first to explore the identification of RGC subtypes within hPSC-derived retinal cultures. This study also provides the foundation for a more comprehensive analysis of hPSC-derived RGCs in future studies, including the investigation of important functional and physiological differences between varying subtypes of RGCs.

The present disclosure provides an important milestone for the differentiation and use of retinal ganglion cells derived from hPSCs by analyzing many of the molecular differences among RGCs, as well as focusing on the major subtypes of these cells that have not been previously studied in humans.

We disclose a highly significant and novel approach to the study of RGCs through the exploration of the diversity and mosaicism of human RGC (hRGC) subtypes. We demonstrated the efficient and conclusive differentiation of each of the major classes of RGC subtypes, and also identified numerous novel molecular markers for these classes of RGCs. The ability to conclusively identify a variety of RGC subtypes, will enable study of hPSC derived RGCs with an understanding of how these subtypes may vary in their differentiation and function, as well as how they are differentially affected in disease states.

In one embodiment differentiated RGC subtypes from human pluripotent stem cells are used for a more detailed analysis of the development of retinal ganglion cells, as well as how different RGC subtypes may be differentially affected in disease states. High throughput screening of drugs and small molecules are used to identify a more directed approach to differentiating individual RGC subtypes, small molecule libraries are used to identify those factors and signaling pathways that lead to increased production of specific RGC subtypes. Such directed differentiation of individual subtypes permits determination of the characteristics and functionality of specific subtypes as well as their susceptibilities in disease states, allowing for differentiation of the effects of different drugs and therapeutics on individual subtypes in diseases which target retinal ganglion cells.

The present disclosure provides an analysis of the diversity of RGCs differentiated from human Pluripotent Stem Cells (hPSCs) and characterized by defined subtypes through the expression of subtype-specific markers. Further investigation of these subtypes was achieved using single cell transcriptomics, confirming the combinatorial expression of molecular markers associated with these subtypes. This analysis also provided insight into previously unreported subtype-specific markers. The results of this study represent the first efforts to explore the derivation of RGC subtypes from hPSCs.

The current disclosure focuses on the specification and identification of retinal ganglion cell (RGC) subtypes differentiated from human pluripotent stern cells using subtype-specific molecular markers. More than 30 different subtypes of RGCs are known to exist in various animal models, with the identification of these varying subtypes based upon the expression of previously established subtype-specific markers. The present disclosure provides the first extensive identification of the major subclasses of RGCs in human-derived cells using subtype-specific molecular markers including direction selective-, alpha-, and intrinsically photosensitive-RGCs.

The present disclosure provides numerous advancements in developmental and disease research. The identification of RGC subtypes using molecular markers reveals numerous aspects off RGC development that have not been explored to date. The use of human pluripotent stem cells as a model system provides a novel and powerful tool to study some of the earliest events in human retinal development. The identification of RGC subtypes using molecular markers early in retinal development can uncover important signaling pathways. In certain embodiments, these markers are used to direct, or select for differentiation of one subtypes over another, with varying subtypes requiring different signaling cues for proper development. Furthermore, as each subtype varies in their functional characteristics, more detailed analyses of individual subtypes as provided by the present disclosure provide for a greater understanding of their specific roles in the visual transduction pathway. In some embodiments the present disclosure provides for methods for treating and/or diagnosing blinding diseases which cause degeneration and eventual death of RGCs. Blinding diseases often appear to preferentially target one RGC subtype over overs. In accordance with the present disclosure, RGC subtypes are identified using molecular markers in disease models of blinding diseases, such as glaucoma, with an emphasis on whether one subtype is predominantly targeted for degenerative effects compared to other subtypes. The ability to investigate the effects of degenerative diseases on specific RGC subtypes allows for a more refined and targeted approach to eventual treatments for optic neuropathies, including the development of pharmaceutical and cell replacement strategies to counter the degenerative processes observed in each subtype.

In certain embodiments single cell RNA sequence analysis is used to identify RGC subtypes in hPSC-derived cells as disclosed herein. With the ability to readily differentiate large quantities of RGCs containing numerous RGC subtypes from hPSCs, individual RGCs can be analyzed using single cell RNA sequence to gain insight into the discovery of novel subtype specific markers. The discovery of these markers can then be used to further characterize and identify different subtypes in culture and increase the understanding of these subtypes from both a developmental and disease standpoints.

The ability to derive RGCs from hPSCs has been the subject of several recent studies, as these cells function to transmit visual information between the eye and the brain, and are functionally compromised in several blinding disorders, with little emphasis upon the diversity of RGCs known to exist. To date, numerous RGC subtypes have been identified within animal models based upon morphological features as well as functional properties, although such subtypes within human RGCs have been much less characterized. The generation of hPSC-derived RGCs in a manner which represents the diversity of RGC subtypes provides a more reliable and realistic model for studying the development of RGCs, with important implications on how individual subtypes may be differentially affected in various disease states. The recent discovery and advancement of molecular markers for specific RGC subtypes facilitates the ability to identify these populations within hPSC-derived RGCs. The results of this study represent the first efforts to extensively investigate and identify the diversity of RGC subtypes within hPSC-derived retinal cells. Furthermore, these studies also contribute to a greater characterization of human RGC subtypes, with results identifying novel molecular markers for major classes of these cells.

RGCs exhibit numerous unique characteristics that set them apart from other retinal cell types, such as their morphological appearance and their transcriptional profile. The expression of many genes can be used to identify RGCs, although the expression of BRN3B, ISL1, SNCG, and RBPMS have been among the most widely utilized molecular markers. Each of these markers was observed within populations of hPSC-derived RGCs, although RBPMS was observed less frequently than the other RGC markers. Traditionally, RBPMS is considered to be a pan-RGC marker, therefore the reduced expression of this gene is a likely indicator that RBPMS is expressed somewhat later than the other RGC markers analyzed. Thus, when studying early stages of RGC development with hPSCs, the use of RBPMS likely underestimates the total RGC population.

SRCCA analysis was used in the current study to confirm the expression of a number of other RGC-associated genes that were highly correlated with these four commonly utilized RGC markers. Applicants surprisingly discovered that the most highly correlated genes associated with each of these four markers revealed striking differences in the transcriptional profiles of these cells, suggesting diversity among the RGC population. Additionally, diversity among RGCs became more apparent when SRCCA analyses from the four target genes were combined for the top 200 genes correlating with all of these markers. These results displayed heterogeneity within RGCs through the differential expression of correlating markers, and this diversity among RGCs could potentially be explained by the presence of numerous subtypes within the RGC population. Thus, the results displayed by single cell RNAseq analysis prompted the exploration of RGC subtypes within hPSC-derived cells.

The varied subtypes of RGCs play important but functionally distinct roles in the visual transduction pathway. Traditionally, efforts have relied largely on dendritic morphology and stratification to identify and characterize RGC subtypes, with different subtypes encompassing unique sizes, shapes, and arborization within the inner plexiform layer. For example, many primate RGCs have often been clustered into categories including midget RGCs or parasol RGCs based upon such morphological features. Such studies have been more complicated for human RGC subtypes, due to a more limited access to the retina for experimental investigation. More recently, the development of molecular markers associated with specific subtypes in rodent models has facilitated the ability to identify these cells more readily. However, such molecular markers remain largely unexplored for primate RGCs, including humans.

The current disclosure represent the first demonstration that molecular markers for major subtype classes could be utilized to readily identify RGC subtypes in hPSC-derived RGCs, representing exciting new avenues for research in RGC diversity. Additionally, the opportunity exists to study species-specific differences in the expression of molecular markers corresponding to RGC subtypes. In the current study, some molecular markers that have been used to identify mouse RGC subtypes (for example, HB9 and HoxD10) were not found to be expressed in hPSC-derived RGCs. While this difference could possibly be due to a degree of maturation not found within the hPSC-derived RGCs examined in the current study. It is also possible that these proteins do not serve as effective molecular markers for RGC subtypes in human cells, highlighting the importance of examining these molecular markers among many different species.

Furthermore, a more enhanced characterization of RGC subtypes was performed in the current study through the analysis of single cell transcriptional profiles, with this exploration providing the ability to identify new molecular markers of RGC subtypes, particularly those for human RGCs. In combination with the RGC specific marker BRN3B, SRCCA analyses performed with multiple RGC subtype-specific genes revealed novel identifiers for major subtype classes. In particular, the combination of BRN3B and SNCG with the direction selective-RGC marker FSTL4 demonstrated a high number of similarly correlated genes, as opposed to a very limited number of correlated genes when FSTL4 was paired with molecular markers of other retinal cell types. As such, this list of genes highly correlated with the expression of BRN3B, SNCG and FSTL4 provides important information about the functionality of these cells, as well as provides new molecular markers for diagnostic purposes. Among these highly correlated genes, Doublecortin (DCX) was identified as an intriguing candidate within the list of genes highly correlated with both FSTL4 and BRN38. Previous work has demonstrated a role for DCX in neurogenesis in the CNS, and its expression has also been observed within the RGC layer in a limited number of studies. In the current study using hPSC-derived RGCs, OGX expression was found to be exclusively expressed within RGCs, although only a small subset of RGCs expressed DCX, indicating the specificity of DCX expression within a given RGC subtype. Additionally, a high degree of correlation was observed between DCX and FSTL4-positive direction-selective RGCs, demonstrating the utility of DCX as a molecular marker for direction selective-RGCs. Thus, the observed expression of DCX is indicative of a particular subset of RGCs, rather than an indicator of their degree of maturation. These results highlight the use of SRCCA for the discovery and validation of additional novel genes expressed within specific RGC subtypes.

The identification of RGC subtypes using molecular markers also reveals numerous aspects of RGC development that have not been explored to date. The use of hPSCs as a model system provides a novel and powerful tool to study some of the earliest events in human retinal development. The identification of RGC subtypes using molecular markers early in retinal development uncovers important signaling pathways that lead to the differentiation of one subtype over another, with varying subtypes requiring different signaling cues for proper development. Furthermore, as each subtype varies in their functional characteristics, more detailed analyses of individual subtypes allows a greater understanding of their specific roles in the visual transduction pathway.

Additionally, blinding diseases which cause degeneration and eventual death of RGCs often appear to preferentially target one subtype over others. The present disclosure provides for techniques including the use of hPSCs (including patient derived hPSCs) to identify RGC subtypes using molecular markers in disease models such as glaucoma, with an emphasis on whether one subtype is predominantly targeted for degenerative effects compared to other subtypes. The ability to investigate the effects of degenerative diseases on specific RGC subtypes allows for a more refined and targeted approach to eventual treatments for optic neuropathies, including the development of pharmaceutical and cell replacement strategies to counter the degenerative processes observed in each subtype.

In certain embodiments the present disclosure also provides for donor plasmids and guide RNAs for creating CRISPR reporters for proteins associates with RGC development, for example Meis2, SPP1, and OPN4. Meris2 is a transcription factor implicated in non-human primate RGC subtypes and may correlate with human RGC subtypes, SPP1 for alpha RGCs, and OPN4 for intrinsically photosensitive RGCs. These new tools allow for determination of differences in developmental timing as well as correlation in culture to different RGC morphologies. The present disclosure encompasses the donor plasmids and guide RNAs as well as the use thereof as outlines herein. The present disclosure also encompasses genetically modified organism which have been modified with a reporter gene for expression of one or more of the proteins and/or markers described herein.

Analyses as disclosed herein may be used for detection of a disease or diseased state affecting RGCs, including glaucoma.

The following specific Examples are provided to promote a further understanding of certain aspects of the present disclosure. It will be understood that these Examples are illustrative, and not limiting, in character.

### Example 1

Human pluripotent stem cells were differentiated to a retinal progenitor fate and then to an early optic cup stage with the development of RGCs occurring around day 40 of differentiation. At this time, free floating cell aggregates, known as retinal organoids, were dissociated for 20 minutes into single cells using accutase with addition of DNAse. Single cells were counted and plated onto poly-ornithine and laminin coated coverslips at a concentration of 50,000 cells/coversllp. RGCs were allowed to mature on coverslips for an additional 40 days. During this time, RGCs were supplemented with BrainPhys Neuronal Media with the addition of B27, N2, dibutryl cAMP, ascorbic acid, brain-derived neurotropic factor (BDNF), and glial-derived neurotropic factor (GDNF), Within 80 days of differentiation, coverslips were fixed with 4% paraformaldehyde and prepared for immunostaining, RGC subtype-specific antibodies were chosen and coverslips were stained followed by analysis of RGCs using immunofluorescence. RGC type antibodies in co-expression with BRN3, the most prominent marker of RGCs were used to identify RGC subtypes including direction selective-, alpha-, and intrinsically photosensitive-RGCs.

Additionally, within 80 days of differentiation, RGCs were dissociated into single cells and analyzed using single cell quantitative reverse transcription polymerase chain reaction (qRT-PCR) which recapitulated the expression of subtype-specific markers as well as demonstrated the combinatorial expression of molecular markers and allowed further characterization of RGCs into a specific category. This disclosure is the first to provide in-depth detail regarding the presence of RGC subtypes in human derived cells and the characterization of these subtypes based on specific molecular markers. Subsequently, single cell RNAseq was also performed on dissociated RGCs to elucidate the RGC subtypes in our hPSC-derived RGCs and provide information into the discovery of additional molecular markers specific for different RGC subtypes. Single cell RNAseq data from RGCs was analyzed using Spearman's Rank Correlation Coefficient Analysls (SRCCA) to explore novel markers for direction-selective RGCs. This type of correlation analysis yielded a gene, DCX, which was highly correlated with other direction selective markers and RGC-associated markers. Further studies using immunocytochemistry, validated the expression of DCX exclusively to RGCs, although only a small subset of RGCs expressed this protein. Our results highlight how SRCCA analysis of single cell RNAseq can be used for the discovery and validation of additional novel genes expressed within RGC subtypes. The discovery of more subtype specific markers is essential in gaining a better understanding of how these RGC subtypes develop in humans as well as how these subtypes may be affected in disease states such as glaucoma.

In regards to previous attempts to differentiate RGCs using The Meyer Lab protocol, numerous changes have been made to allow for the maturation and identification of RGC subtypes. Changes to previous protocol include a gentler handling and dissociation of the retinal cells into single cells (previously partial dissociations were used instead of dissociations into single cells), the concentration retinal cells were plated at following dissociation (previously this concentration was much higher which caused dumping and an inability to identify and study individual cells), and lastly the use of BrainPhys media with the components and growth factors listed above (Retinal differential media was previously used). The changes listed above have increased the level of maturation of our hPSC-derived RGCs, allowing us to identify RGC subtypes readily in our cultures. Most notably, previous efforts of the lab grew RGCs using Retlnal Differentiation Media containing B27, which caused prolonged proliferation, and limited the ability of h1PSC-derived RGCs to mature and differentiate into specific RGC subtypes.

### Example 2

### Cellular Diversity of Retinal Ganglion Cells Derived from Human Pluripotent Stem Cells

As noted above, RGCs serve as the functional connection between the eye and the brain, extending lengthy axons vital for the transmission of visual information. As such, the ability to derive RGCs from hPSCs has been an increasingly important field of research in recent years, with the potential to use these cells not only for cellular replacement, but also for their use in disease modeling and pharmacological screening. Previous studies that have investigated the derivation of retinal cells from hPSCs have largely focused upon these populations as a whole, with the identification of RGCs relying solely upon the expression of BRN3, a transcription factor specific to such cells in the retina. However, as the projection neurons of the retina, RGCs exhibit numerous morphological, phenotypic, and functional differences associated with the roles they play in visual transduction. Thus, initial efforts were undertaken to examine hPSC-derived RGC populations for differences in their transcriptional profiles to identify genes that may constitute a core network of genes associated with a variety of RGC subtypes, as well as those that may be expressed in subsets of RGCs.

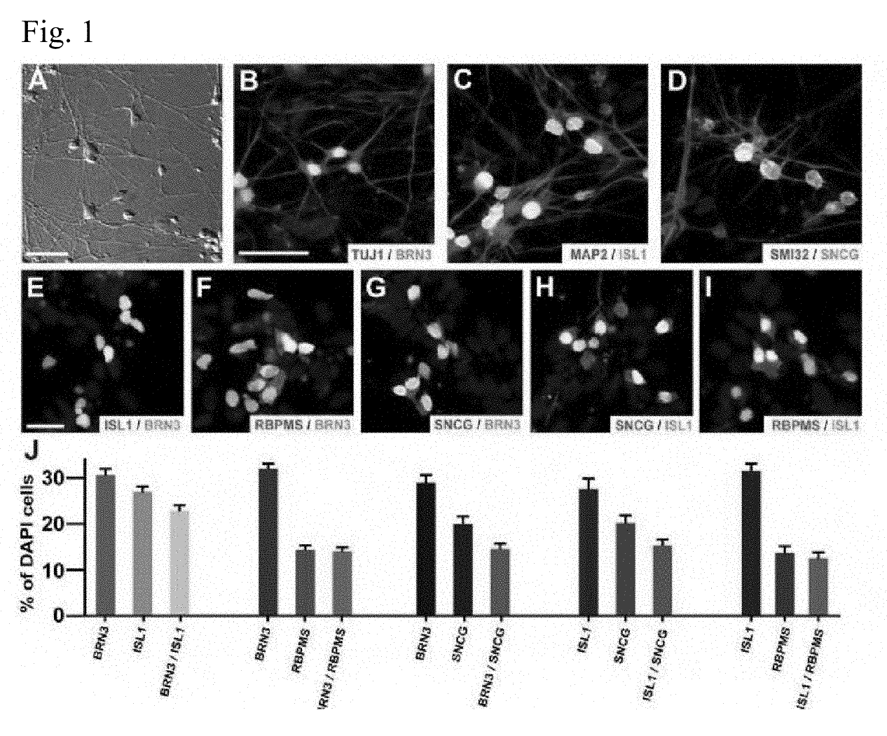

hPSC-derived RGCs exhibited elaborate morphological features including the extension of lengthy neurites within the first 80 days of differentiation (FIG. 1). These RGCs exhibited numerous characteristic features, with large three-dimensional somas interconnected by long neuronal processes (FIG. 1A), The RGC fate of these cells was confirmed by the expression of RGC-associated proteins, including RGC-associated transcription factors such as BRN3, ISL1 and SNCG, as well as long and interconnecting neurites expressing a variety of characteristic cytoskeletal markers (FIG. 1B-D). More so, the diversity among RGCs was demonstrated by co-staining four of the most common RGC associated markers and indicated a variation in expression levels throughout the RGC population (FIG. 1E-J). The resultant RGCs were found to express a complete profile of RGC-associated features and demonstrated a unique diversity in RGC-associated gene expression.

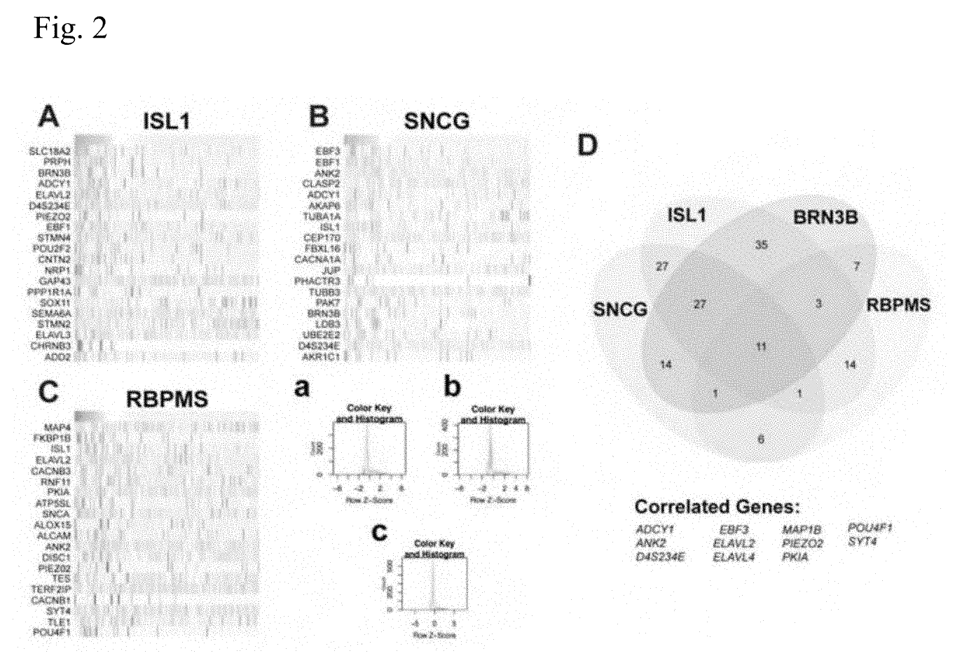

The transcriptional profiles of these hPSC-derived RGCs were analyzed by single cell RNA-seq, with numerous genes found to be expressed in all RGCs while others were expressed within subsets of the complete RGC population (FIG. 2). Spearman's Rank Correlation Coefficient Analysis (SRCCA), a technique used to find genes that correlate most closely with one or more designated target genes across the entirety of a single cell RNA-seq (scRNA-seq) dataset, was used to elucidate single cell data. Commonly studied genes associated with RGCs were used as target genes and SRCCA was performed to identify those genes whose expression was strongly correlated with the expression of these targets across tile entire scRNA seq dataset (FIG. 2A-C). Results of these analyses revealed evident clustering of expression patterns within RGCs.

More so, SRCCA correlations from multiple target genes were combined to identify novel genes specific to a given cell type. To identify unique RGC markers, SRCCA identified the 200 genes most strongly correlating with BRN3B, ISL1, SNGG, and RBPMS. This analysis revealed 11 genes strongly correlated with each of the four target genes (FIG. 2D), including several with known roles predominantly in neuronal cell types. This analysis not only revealed numerous similarities in RGC gene expression but also indicated a distinct heterogeneity within this population of cells in which numerous genes were found to be correlated with only some of the target genes. Thus, these differences were explored to identify the presence of distinct RGC subtypes within the greater hPSC-derived RGC population.

### Example 3

### Identification of Defined RGC Subtypes within hPSCs-derived Cultures

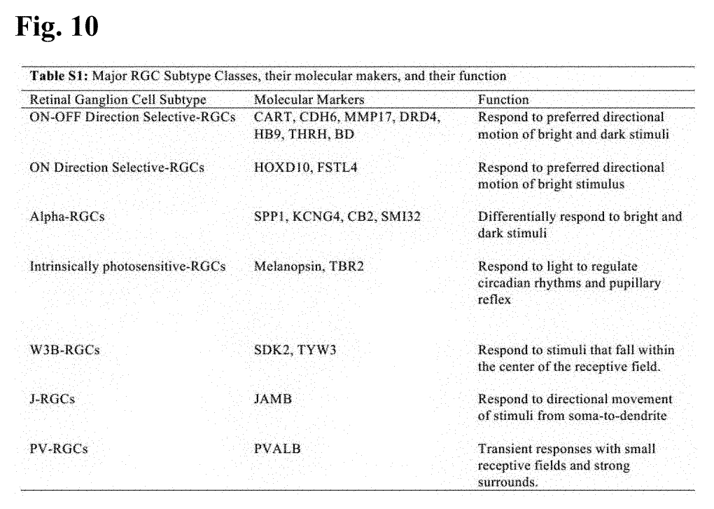

More than 30 different subtypes of RGCs are known to exist, varying in their gene expression patterns, morphological features, and functionality. Given the variability in gene expression observed among hPSC derived RGCs (FIG. 2), efforts were undertaken to further examine the specific RGC subtypes present within these cultures based on the expression of genes characteristic for each class (Table S1, FIG. 10). Direction selective-RGCs (DS-RGCs) constitute a group of cells capable of responding to preferred directional motion in response to bright and dark stimuli, with the identification and functionality of these cells previously explored in animal models. However, such cells have not been definitively identified in human RGCs. DS-RGCs have been divided into two groups: ON-OFF DS-RGCs capable of detecting preferred directional motion to bright and dark stimuli, and ON DS-RGCs which respond to the directional motion of a bright stimulus. Each category of DS-RGC has been identified by the expression of specific molecular markers, such as CART, CDH6, and FSTL4, and further characterized into subgroups based upon preferred directional motions (dorsal, ventral, nasal and temporal) through the combinatorial expression of specific genes.

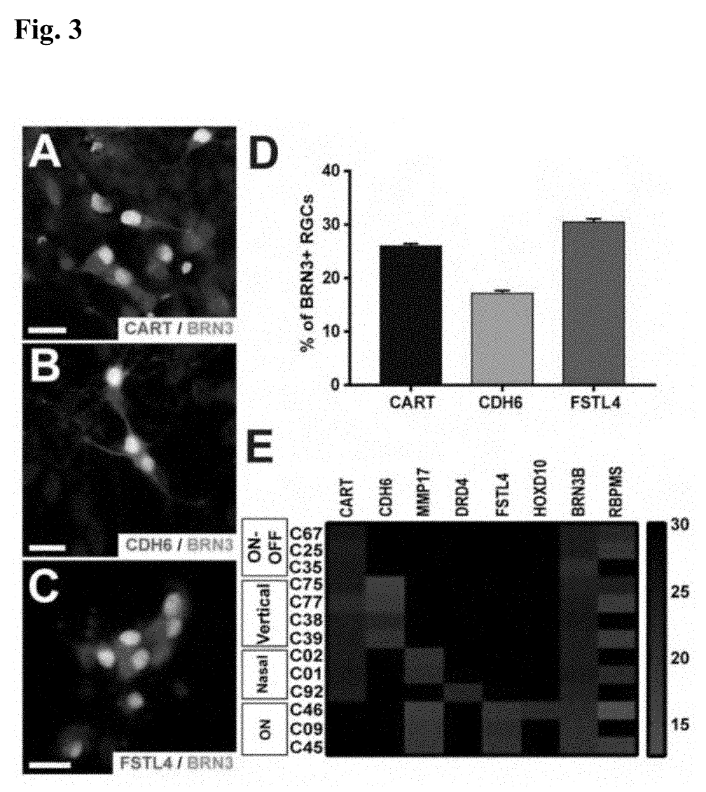

DS-RGC-like cells were identified within hPSC cultures within tile first 80 days of differentiation, with the expression of BRN3 serving as confirmation of their RGC lineage. Presumptive ON-OFF DS-RGCs were identified based upon their expression of CART in 25.95%±0.46% of the BRN3-positive RGC population (FIG. 3A, D). DS-RGCs were further divided into ON-OFF as well as ON subgroups, identifiable based upon the expression of genes such as CDH6 and FSTL4, respectively. Thus, a more detailed analysis of hPSC derived RGCs revealed that CDH6-positive ON-OFF DS-RGC-like cells were identified within 17.16%±0.51% of the BRN3-positive RGC population (FIG. 38, D). Similarly, the expression of FSTL4, indicative of ON DS-RGCs, was found within 30.49±0.58% of BRN3-positive RGCs (FIG. 3C, D). As the combinatorial expression of several genes is often required for the identification of some RGC subtypes, single cell qRT-PCR confirmed the combined expression of numerous DS-RGC markers within individual RGCs (FIG. 3E).

Alpha-RGCs represent another RGC subtype which vary in their response to bright and dark stimuli with their responses, including transient OFF, sustained OFF, and ON. Previous studies have demonstrated high levels of Osteopontin (SPP1) expressed in alpha-RGCs, with this gene currently serving as the main identifier of this class of cells. Additional markers such as KCNG4 and CB2 have also been identified to further classify alpha-RGCs. Thus, the presence of alpha-RGCs within the hPSC-derived RGC population was investigated using these genetic markers.

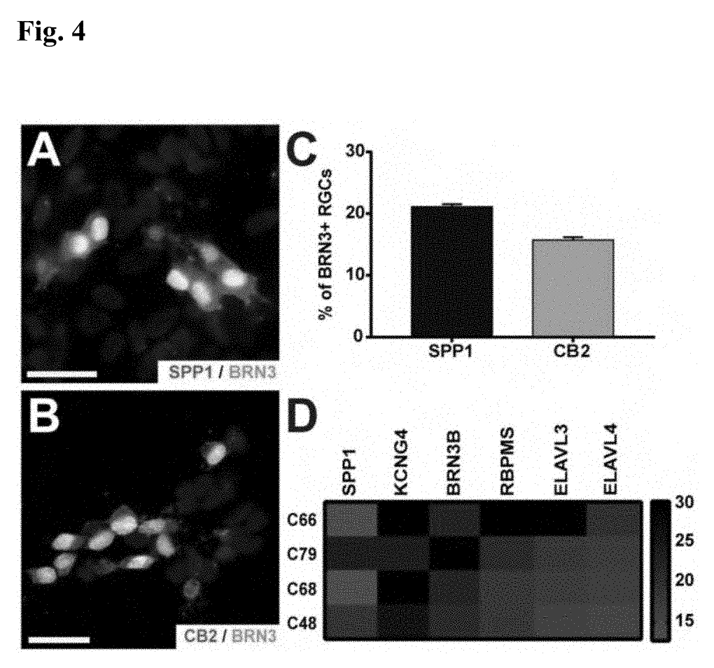

Presumptive alpha-RGCs were identified in hPSC retinal cultures by the combinatorial expression of alpha RGC-associated genes in conjunction with the pan-RGC marker, BRN3. SPP1 expression was discovered in 21.10%±0.42% of the BRN3-positive RGCs (FIG. 4A, C). Further analysis demonstrated the expression of CB2, which identifies a subset of alpha-RGCs with the capability of having a transient response to dark stimulus, co-localized in 15.72%±0.45% of BRN3-RGCs (FIG. 4B, C). More so, single cell qRT-PCR analyses confirmed the combinatorial expression of SPP1 and KCNG4 thin individual RGCs, characteristic of alpha-RGCs, in addition to the expression of a variety of other RGC-associated markers (FIG. 4D).

Intrinsically photosensitive RGCs (ipRGCs) constitute an additional RGC subtype, with the unique ability to directly respond to light stimuli with the photopigment Melanopsin. Various subtypes of ipRGCs have been shown to control aspects of circadian rhythm as well as pupillary reflexes by projecting to various non-image processing areas in the brain. Previous studies have discovered ipRGC subcategories (M1-M5), which vary in their genetic signature, light response, and physiological functions, although studies of human ipRGCs have been more limited. As such, a detailed analysis was performed to explore the presence and transcriptional profile of 1pRGG-like cells within the hPSG-derived RGC population.

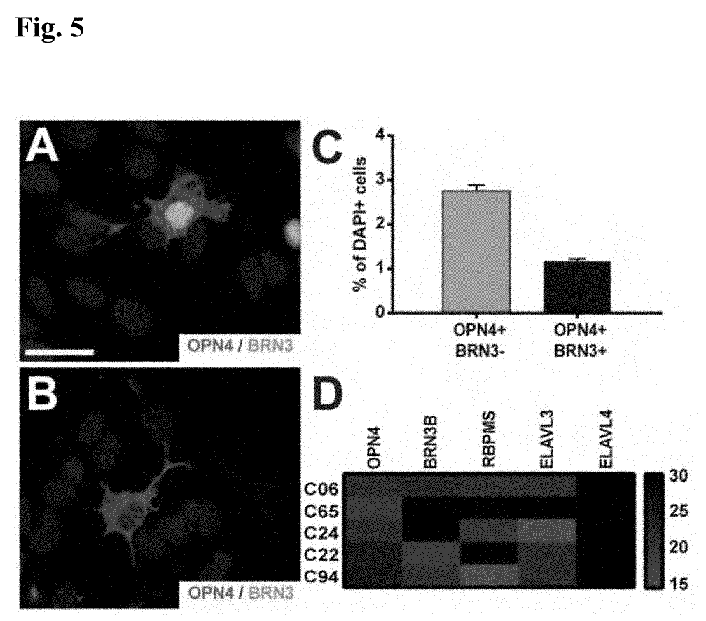

Melanopsin was expressed either with or without the co-expression of BRN3, consistent with previous studies that have demonstrated BRN3 expression confined to only some types of ipRGCs. This Melanopsin expression was observed in 1.17%±0.06% of all cells in conjunction with BRN3, while 2.75%±0.13% of differentiated retinal neurons expressed Melanopsin in the absence of BRN3 (FIG. 5C). Single cell transcriptional analyses verified the expression of Melanopsin within individual RGCs in combination with BRN3, as well as with the combinatorial expression of other RGC-related markers (FIG. 5D).

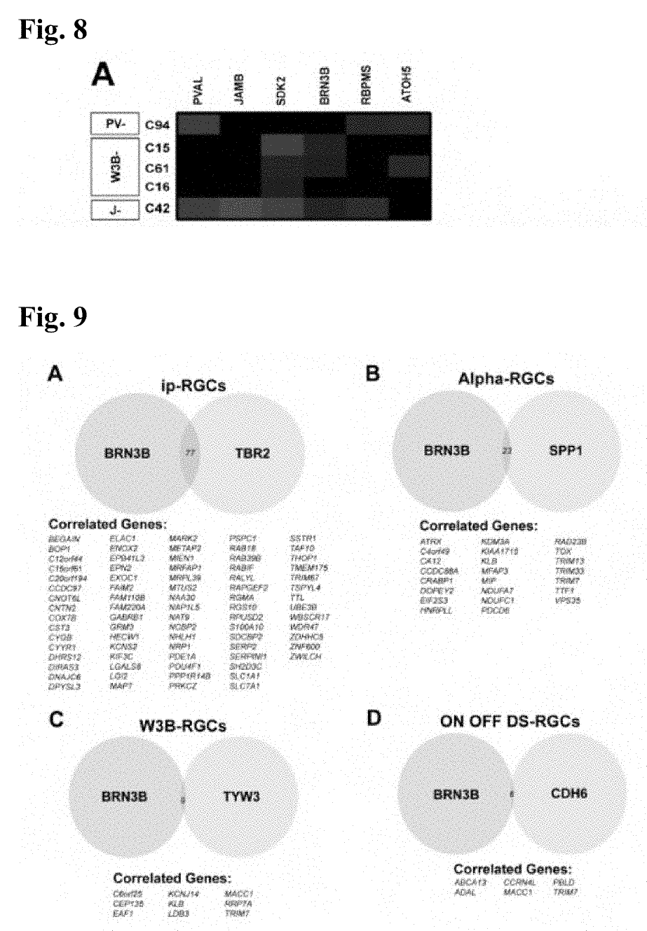

Furthermore, a variety of other RGC subtypes have been previously identified including PV-, W3B-, and J-RGCs. Single cell qRT-PCR analyses demonstrated the expression of Parvalbumin, SDK2, and JAM-B which indicated the presence of such subtypes within hPSC-derived cultures, respectively (FIG. 8).

### Example 4

### Investigation of Novel Molecular Markers for DS-RGCs

The ability to reliably identify specific RGC subtypes based on tile expression of molecular markers has only recently been described with a relatively limited set of markers associated with these subtypes (Table S1, FIG. 10). The investigation of additional molecular markers strongly correlated with these subtypes would not only provide for a greater ability to identify specific types of cells, but could also provide some insight into differences in functionality between these cells. SRCCA approaches as detailed herein provide the power to elucidate transcriptional differences between individual cell types and as such, provide an advantageous tool for the identification of novel molecular markers associated with individual RGC subtypes. As DS-RGCs were among the most prevalent subtypes identified within cultures of hPSC-derived RGCs (FIG. 3C), efforts were made to identify those genes that were most closely correlated with this phenotype. Additional analyses were also performed to identify those genes most strongly correlated with markers of additional RGC subtypes (FIG. 9).

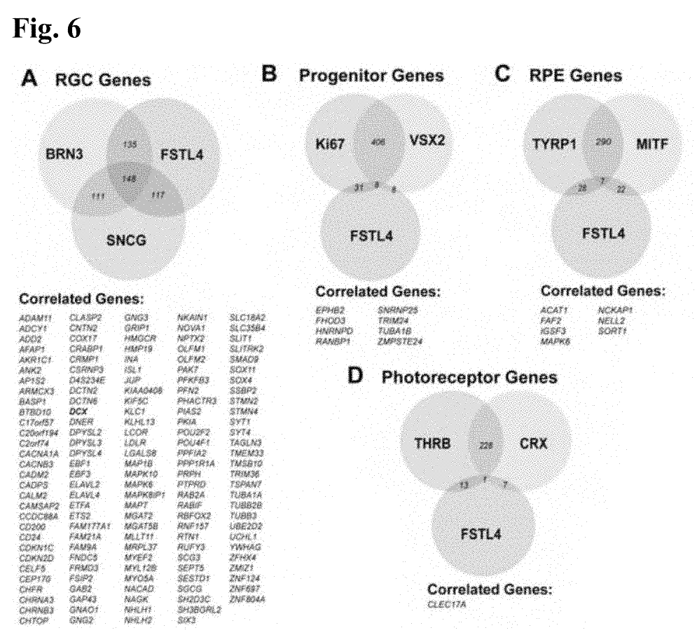

For analysis of DS-RGCs, SRCCA was performed to identify those genes that were most strongly correlated with the expression of FSTL4, BRN3B and SNCG, with numerous genes identified to be strongly correlated with each of the three target genes (FIG. 6A). Furthermore, combined SRCCA was performed with FSTL4 and genetic markers for retinal progenitors, RPE, and photoreceptors. Overlap between FSTL4 and markers for each of these latter cell types was minimal, indicating a strong degree of specificity for FSTL4 expression in RGCs (FIG. 6B-D). The results of this analysis provided a total of 148 genes which could potentially serve as new genetic identifiers for DS-RGCs. Of these genes, doublecortin (DCX) was further explored as one of the 148 genes strongly correlated between the three target genes. Previous studies have identified a role for DCX in early neurogenesis of the central nervous system, however its pattern of expression in the retina has not been studied in great detail.

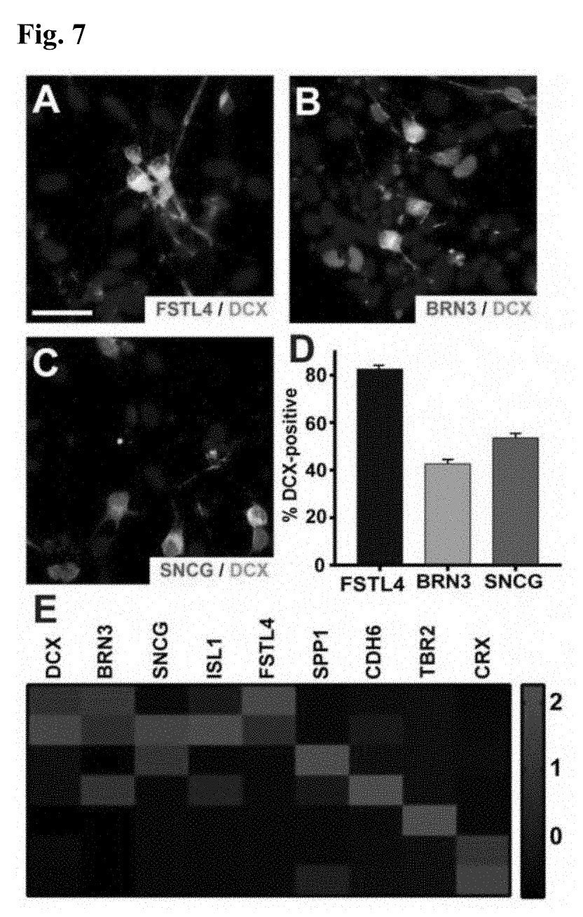

The association of DCX with a specific subtype of RGC, namely DS-RGCs, was further investigated in hPSC-derived cells. Immunocytochemistry results revealed DCX expression to be highly co-expressed with DS-RGC markers such as FSTL4 (FIG. 7A), but only in a subset of BRN3- and SNCG-expressing RGCs (FIG. 7B-C). BRN3-expressing RGCs co-immunostained for DCX in 42.61%±1.88% of the population and SNCG-positive RGCs expressed DCX in 53.57%1±1.88% of the RGCs. More so, quantification revealed that FSTL4-positive RGCs co-localized with DCX at 82.48%±1.66% (FIG. 7D). Additionally, single cell RNAseq demonstrated tile specificity of DCX expression with DS-RGCs apart from other RGCs and retinal cell types (FIG. 7E). Thus, the results of this analysis have identified DCX as a potentially novel and useful marker for this important subset of RGCs.

### Example 5

### Maintenance of hPSCs

Human pluripotent stem cell lines miPS2, H7 and H9 were grown and maintained in their pluripotent state following previously described protocols (Meyer et al., 2011; Meyer et al., 2009; Ohlemacher et al., 2015). Briefly, hPSCs were maintained upon matrigel-coated six well plates in mTeSR1 medium (Stem Cell Technologies). Cells were passaged upon reaching approximately 70% confluency. Undifferentiated cells were examined before passaging for areas of spontaneous differentiation, and such areas were marked and mechanically removed. Colonies were then enzymatically lifted using dispase for approximately 15 minutes, and hPSCs were then replated at a 1:6 ratio.

### Example 6

### Differentiation of hPSCs

Retinal differentiation of hPSCs was accomplished using newly developed protocols. Differentiation was initiated via the formation of embryoid bodies, which were then transitioned over a 3 day period from mTeSR1 medium to Neural Induction Medium (NIM: Dubelco's Modified Eagle Medium (DMEM)/F12 (1:1), M2 supplement, Minimal Essential Medium (MEM) nonessential amino acids, heparin (2 mg/ml), and Penicillin-Streptomycin). After a total of 7 days of differentiation, embryoid bodies were induced to adhere to a six well plate using 10% Fetal Bovine Serum (FBS) in NIM overnight. The next day, FBS was removed and cells were grown in NIM until day 16, with a media change every other day. At this time point, colonies were mechanically lifted and transferred into suspension culture as cell aggregates in Retinal Differentiation Medium (RDM: DMEM/F12 (3:1), MEM nonessential amino acids, B27 supplement, and Penicillin-Streptomycin). Suspension cultures yielded the formation of both retinal organelles and non-retinal forebrain neurospheres, with the retinal organoids identified and isolated based on their morphological characteristics within 30 days of differentiation. After a total of 40 days of differentiation, retinal organoids were dissociated into a single cell suspension using Accutase and plated on coverslips at a concentration of 50,000 cells per 12 mm coverslip. Cells were maintained up to 80 days of differentiation using BrainPhys Neuronal Media, with a media change twice a week.

### Example 7

### Immunocytochemistry

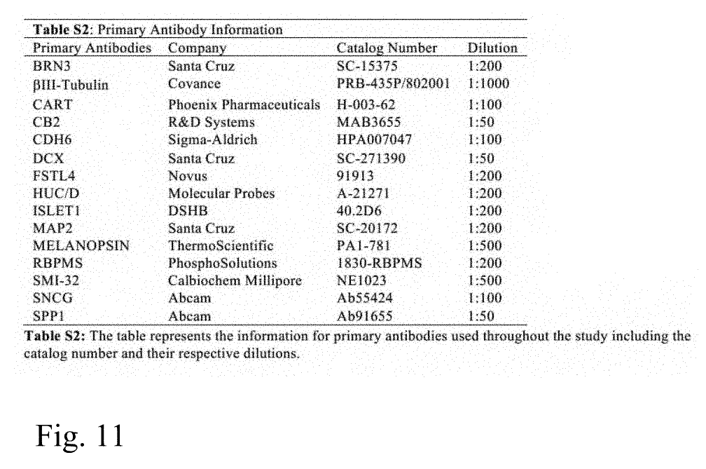

Cultures of retinal ganglion cells at 80 days of differentiation were fixed with 4% paraformaldehyde in phosphate buffer solution (PBS) for 30 minutes at room temperature. Samples were then washed 3 times with PBS, followed by permeabilization using 0.2% Triton X-100 solution for 10 minutes. Cells were then blocked with 10% donkey serum at room temperature for 1 hour. Primary antibodies (Table S2, FIG. 11) were diluted in 5% donkey serum and 0.1% Triton X-100 and applied to cells overnight at 4° C. The following day, cells were washed 3 times with PBS and blocked with 10% donkey serum for 10 minutes. Secondary antibodies were diluted at a concentration of 1:1000 in 5% donkey serum and 0.1% Triton X-100 and added to cells for 1 hour at room temperature. Cells were then washed 3 times with PBS before mounting onto slides for microscopy.

### Example 8

### Microscopy and Data Quantification

Following immunocytochemical staining, retinal ganglion cells were imaged using a Leica DM5500 fluorescence microscope. Four regions from multiple coverslips were imaged for the expression of the RGC marker BRN3 and various subtype markers, and these experiments were replicated three times using separately differentiated batches of cells. The number of RGCs were quantified based upon BRN3 expression using Image-J cell counter plugin, and subtype markers were quantified according to their co-expression with BRN3. The average number of cells expressing each subtype marker in conjunction with BRN3 was quantified along with the standard error of the mean, and statistically significant differences were determined using GraphPad Prism software.

### Example 9

### C1 Single Cell qRT PCR Data Collection and Analysis

Within 80 days of differentiation, hPSC-derived RGCs were dissociated into single cells with accutase for 10 minutes. Single cells were sorted using the SORP Aria cell sorting machine. 10,000 cells were loaded into capture sites of the C1 Single-Cell Auto Prep fluidic circuit After loading, individual capture sites from the C1 plate were examined and the contents of each capture site was recorded (0 cell, 1 cell, more than 1 cell, or debris). Reverse transcription and cDNA amplification was completed on each capture site of the C1 circuit. The amplified cDNA was then loaded into the Biomark qRT-PCR machine with pre-designed RGC-specific and RGC-subtype primers. Any values from wells with zero or more than one cell were discarded in the analyses. The Ct values from the Biomark qRT-PCR analysis were sorted based on the expression of BRN3B and their expression levels were further constructed into heat maps of specific RGC subtype classes using GraphPad Prism software.

### Example 10

### Single Cell RNAseq Data Collection and SRCCA Analysis

SRCCA was performed using published scRNA-seq datasets from day 70 hPSC retinal cultures (CRX^{+tdTomato} reporter line created on a H9 hESC line background [Wicell], n=3), and detailed methods for single cell capture, cDNA preparation and library generation, sequencing, read mapping, quality control, and gene expression quantification were previously reported. Phillips, et al., A Novel Approach to Single Cell RNA-Sequence Analysis Facilitates In Silico Gene Reporting of Human Pluripotent Stem Cell-Derived Retinal Cell Types (2017, Dayton Ohio), incorporated herein in its entirety. Briefly, cell capture and library preparations were performed with the Fluldfgm C1 system according to manufacturer's instructions. cDNA was generated with the SMARTer PCR cDNA Synthesis kit (Clontech) and amplified with the Advantage 2 PCR kit (Clontech). Single cell cDNA libraries were fragmented and amplified with the Nextera XT DNA sample preparation and index kit (lllumina), multiplexed (24-48 libraries per lane) and 51-bp single-end reads were sequenced (Illumina HiSeq 2500 System). FASTQ files were generated by CASAVA (v1.8.2) and mapped using Bowtie (v0.12.8). RSEM (v1.2.3) was used to calculate normalized gene expression values in TPM (transcripts per million). Quality control analysis was performed using the SinQC program. SRCCA analysis of RGC target genes was performed by R programming language. Correlating genes were ranked based upon Spearman's correlation coefficients (rho) from high to low values. To determine overlap between SRCCA lists from multiple target genes, gene lists were imported into Venny 2.1 or Meta-Chart Venn diagram generator.

FIG. 1: hPSC-derived RGCs display elaborate morphologies and diversity of gene expression. (A) DIC imaging demonstrated morphological characteristics of hPSC-derived RGCs with large three-dimensional cell bodies, projecting numerous lengthy neurites. (B-D) immunocytochemistry confirmed the RGC identity of these cells through the expression of specific markers such as BRN3, ISL1, and SNCG, as well as the extension of long processes indicated by cytoskeletal markers. (E-1) Additionally, analysis of RGC markers revealed varying degrees of co-expression within the RGC population, (J) Quantification of immunocytochemistry results verified variation in RGC-associated gene expression among hPSC-derived RGCs. Scale bars equal 50 μm in A-D, 25 μm in E−1, scale bar in B applies to B-D, scale bar in E applies to E-1. Error bars represent standard error of the mean (n=27 technical replicates from 3 biological replicates for each bar using miPS2, H9, and H7 cell lines).

FIG. 2: hPSC derived RGCs demonstrate unique transcriptional profiles. (A-C) Spearman's Rank Correlation Coefficient Analysis (SRCCA) was performed on single cell RNAseq data using RGC-specific target genes. Cells were ranked from highest to lowest expression and transformed into z-scores. Results were constructed into a heat map, with the top 20 correlating genes shown for each RGC marker, (Corresponding color key histograms for A-C are displayed in a-c). (D) The combination of SRCCA from four RGC target genes for the top 200 correlating genes revealed differential gene expression as well as a core set of 11 genes highly expressed within RGCs. n=3 biological replicates using the H9 cell line.

FIG. 3: hPSCs give rise to multiple DS-RGC subtypes. (A-B)ON-OFF DS-RGCs were identified by the co-expression of the RGC-marker BRN3 with either CART or CDH6. (C) ON DS-RGCs were identified by the expression of FSTL4 co-localized with BRN3. (D) Quantification of immunocytochemistry results identified the expression of CART, CDH6, and FSTL4 within the BRN3-RGC population at 25.95%±0.46%, 17.16%±0.51%, and 30.49%±0.58%, respectively. (E) Single cell qRT-PCR analysis demonstrated the combinatorial expression of DS-RGC markers, in conjunction with RGC-associated markers. Error bars represent the standard error of the mean (n=36 technical replicates from 3 biological replicates for each bar using miPS2, H9, and H7 cell lines). Scale bars equal 20 μm.

FIG. 4: Identification of alpha RGCs in hPSC-derived RGCs (A-B) Alpha-RGCs were identified by the co-localization of BRN3 with either SPP1 or CB2. (C) Quantification of immunocytochemistry results indicated SPP1 and CB2 were co expressed with BRN3 in 21.10%±0.42% and 15.72%±0.45% of all cells, respectively. (D) Single cell qRT-PCR analyses demonstrated the combinatorial expression of alpha RGC markers, along with the expression of pan-RGC markers. Error bars represent the standard error of the mean (n=36 technical replicates from 3 biological replicates for each bar using miPS2, H9, and H7 cell lines). Scale bars equal 30 μm.

FIG. 5: Characterization of intrinsically photosensitive-RGCs derived from hPSCs (A-B) A subset of hPSC-derived cells exhibited the expression of Melanopsin co-expressing either with or without BRN3. (C) Quantification of immunocytochemistry results was performed as a percentage of the total DAP1-positive population and Melanopsin-positive/BRN3-negative cells comprised 2.75%±0.13% of all cells, while Melanopsin-positive/BRN3-positive comprised 1.17%±0.06% of all cells. (D) Single cell qRT-PCR revealed the combinatorial expression of Melanopsin with a variety of other RGC related markers, Error bars represent the standard error of the mean (n=36 chemical replicates from 3 biological replicates for each bar using miPS2, H9, and H7 cell lines). Scale bar equals 50 μm.

FIG. 6: Identification of novel DS-associated markers using single cell RNAseq analysis. (A) SRCCA from FSTL4, BRN3B, and SNCG were combined for the top 1000 correlating genes and 148 genes were found to be commonly expressed between the three populations. (B-D) Additionally, SRCCA for FSTL4 was combined with retinal progenitor genes, RPE genes, and photoreceptor genes and demonstrated minimal overlapping expression. n=3 biological replicates using the H9 cell line.

FIG. 7: Identification and confirmation of DCX as a novel DS-RGC marker. (A-C) DCX was highly co-localized with FSTL4, while its co-expression with pan-RGC markers BRN3 and SNCG demonstrated less correlation. (D) Quantification of immunocytochemistry results indicated that DCX expression correlated with 82.48%±1.66% of FSTL4-positive RGCs, while it was identified in subsets of BRN3- and SNCG-positive RGCs at 42.61%±1.88% and 53.57%±1.88%, respectively. (E) Single cell RNAseq values demonstrate expression of DCX correlated with other DS-RGC markers, but was found exclusive of markers of other RGC subtypes and retinal cells. Scale bars equal 50 μm. Error bars represent standard error of the mean (n=30 technical replicates from 3 biological replicates for each bar using miPS2, H9, and H7 cell lines).

FIG. 8: Single cell qRT-PCR reveals expression of markers for other RGC subtypes. A variety of other subtypes were characterized using single cell qRT-PCR. (A) PV-, W3B-, J-RGCs were identified through the expression of PVALB, SDK2, and JAMB respectively, in addition to their combinatorial expression with a variety of other RGC-associated markers.

FIG. 9: Single cell RNA-seq analyses indicate correlative genes for major RGC subtype classes. Single cell RNAseq analyses of numerous subtypes reveals closely correlated genes. (A-D) SRCCA for BRN3B using the top 1000 correlating genes was combined with SRCCA for subtype specific markers and revealed correlated genes. For ip-. Alpha-, W3B-, and On OFF DS-RGCs 77, 23, 9, and 6 genes were found closely correlated within the top 1000 genes.

What is claimed is:

1. A method for diagnosing a disease or diseased state related to retinal ganglion cells in a patient, the method comprising the step of detecting in a body fluid of the patient one or more markers associated with the disease or diseased state.

2. The method of claim 1, wherein the patient is a mammal.

3. The method of claim 2, wherein the patient is human.

4. The method of claim 1, wherein the marker indicates ON-OFF direction selective retinal ganglion cells.

5. The method of claim 4, wherein the marker comprises BRN3 co-expressed with at least one of CART or CDH6.

6. The method of claim 1, wherein the marker indicates ON direction selective retinal ganglion cells.

7. The method of claim 6, wherein the marker comprises FSTL4 co-localized with BRN3.

8. The method of claim 1, wherein the marker indicates alpha retinal ganglion cells.

9. The method of claim 8, wherein the marker comprises BRN3 co-localized with at least one of SPP1 or CB2.

10. The method of claim 1, wherein the marker indicates intrinsically photosensitive retinal ganglion cells.

11. The method of claim 10, wherein the marker comprises Melanopsin.

12. The method of claim 1, wherein the marker comprises DCX.

13. The method of claim 12, wherein the marker comprises DCX co-localized with FSTL4.

14. A method for detection of retinal ganglion cells in a cell culture, the method comprising the steps of:
preparing a cell culture of human pluripotent stem cells; and
detecting one or more markers for retinal ganglion cells in the cell culture.

15. The method of claim 14, wherein the marker comprises BRN3 co-expressed with at least one of CART or CDH6.

16. The method of claim 14, wherein the marker comprises FSTL4 co-localized with BRN3.

17. The method of claim 14, wherein the marker comprises BRN3 co-localized with at least one of SPP1 or CB2.

18. The method of claim 14, wherein the marker comprises Melanopsin.

19. The method of claim 14, wherein the marker comprises DCX.

20. The method of claim 19, wherein the marker comprises DCX co-localized with FSTL4.
